(19) United States
(12) Patent Application Publication
(10) Pub. No.: US 2006/0090247 A1
(43) Pub. Date: May 4, 2006

(54) Visor cap

(75) Inventor: Byoung-Woo Cho, Yongin-city (KR)

Correspondence Address: STAAS & HALSEY LLP, SUITE 700, 1201 NEW YORK AVENUE, N.W., WASHINGTON, DC, 20005, US

(73) Assignee: Yupoong, Inc., Seoul (KR)

(21) Appl. No.: 10/979,136

(22) Filed: Nov. 3, 2004

(51) Int. Cl. A61F 9/00

(52) U.S. Cl. 2/209.3; 2/12

(57) ABSTRACT

A visor cap has a visor portion, a forehead shield coupled to the visor portion, a sweatband formed in shape of a ring to encircle a head of a wearer. The forehead shield has an outer sheet and an inner sheet between which the fore part of the sweatband disposed. At least a part of the sweatband is stretchable to adjust the size of the visor cap to the wearer.

### BACKGROUND OF THE INVENTION

(a) Field of the Invention

The present invention relates to a visor cap, and more particularly, to a visor cap to be fitted onto a head of a wearer regardless of the head size.

(b) Description of the Related Art

Generally, a conventional visor cap is used for shading or protecting the face from light. The visor cap which is adjustable to fit heads of various sizes has a visor portion, a forehead shield, and a stretchable sweatband. The stretchable sweatband may be positioned around the circumference of head so that the visor cap can be fitted regardless of the size of the head. However, the conventional visor cap may produce uncomfortable sensations of compression because the stretchable sweatband excessively presses the head of a wearer. Further, the fore part of the sweatband is stitched to a lower hem of the forehead shield around the visor potion, resulting uncomfortable sensation of the forehead of the wearer. Since the stretchable sweatband is in direct contact with the head of the wearer, the wearer may also experience some discomfort.

### SUMMARY OF THE INVENTION

In view of the prior art described above, it is an object of the present invention to provide a visor cap which is adjustable to fit heads of varying sizes while improving wearing comfort with a soft and comfortable fit.

It is another object of the present invention to provide a visor cap which can be fitted onto heads of various sizes with a simple attractive appearance.

In an embodiment a visor cap may include a visor portion which may be used to block sunlight. A forehead shield with an outer and an inner sheet may be coupled to the visor portion. A sweatband may be placed in the front and rear of the visor cap and is formed in shape of a ring to encircle a head of the wearer. At least a part of the sweatband is stretchable.

The sweatband may include a back strap and an elastic fore strap. The elastic fore strap and the back strap may be placed in the front and rear of the visor cap, respectively. A fore strap may be elastic and include a first end and a second end which are connected to a first end and a second end of a back strap, respectively, to encircle a head of the wearer. The fore elastic strap may be positioned between the outer and the inner sheet.

The elastic fore strap is preferably concealed within the forehead shield at rest or when stretched.

Preferably, the elastic fore strap is fixed to the forehead shield on one or more spots.

### BRIEF DESCRIPTION OF THE DRAWINGS

In the drawings, like elements are depicted by like reference numerals. The drawings are briefly described as follows:

FIG. 1 is a perspective view of a visor cap of the present invention;

FIG. 2 is a side view of the visor cap wherein a portion of an outer sheet of a forehead shield has been broken away to reveal an elastic fore strap;

FIG. 3 is another perspective view of the visor cap;

FIG. 4 is a view wherein an inner sheet is removed in the visor cap of FIG. 3;

FIG. 5 is a perspective view of a back strap coupled to an elastic fore strap; and

FIGS. 6A-6B are sectional views illustrating examples of a forehead shield in which the elastic fore strap is inserted.

### DETAILED DESCRIPTION OF THE PREFERRED EMBODIMENTS

Embodiments of the present invention will hereinafter be described in detail with reference to the accompanying drawings.

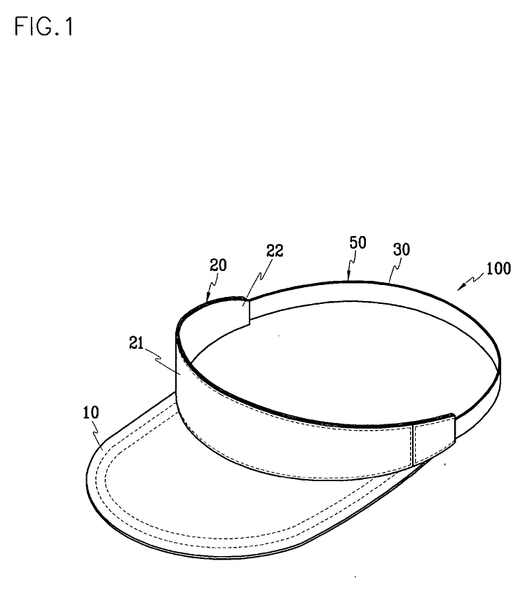

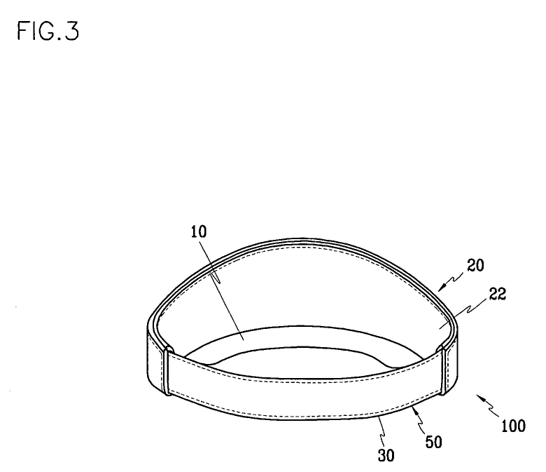

Referring to FIGS. 1 and 3, a visor cap 100 has a visor portion 10, a forehead shield 20 and a sweatband 50. The visor portion 10 is coupled to the forehead shield 20 to block the sunlight from the eyes of a wearer.

The forehead shield 20 has an outer sheet 21 and an inner sheet 22. The outer sheet 21 may be composed of a plurality of pieces according to a desired design, as shown in drawings. It may also be made of a single piece. Similarly, the inner sheet 22 may be composed one or more pieces. When the outer sheet 21 or inner sheet 22 is made of a plurality of pieces, the forehead shield 20 may have an improved contact with the forehead of the wearer. In addition, an interlining may be attached to the outer sheet 21 of the forehead shield 20 in order to retain the shape of the forehead shield 20. The inner sheet 22 is preferably made of a soft material to achieve soft fit as well as rapid sweat absorption.

The sweatband 50 is formed in shape of a ring to encircle a head of a wearer. At least a part of the sweatband is stretchable to be fitted onto a head of a wearer regardless of the head size.

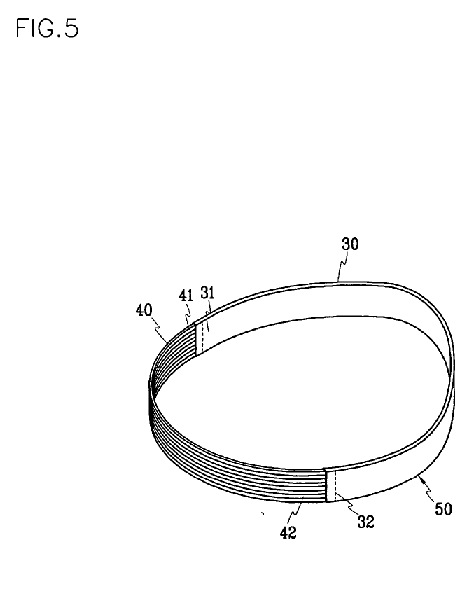

In the embodiment the sweatband 50 is composed of a back strap 30 and a fore strap 40, as shown in FIG. 5. The back strap 30 is connected to an elastic fore strap 40 to form a single ring. The elastic fore strap 40 has a first end 41 and a second end 42 which are connected to a first end 31 and a second end 32 of the back strap 30, respectively. When the elastic fore strap is coupled to the back strap the resulting ring may encircle the head of the wearer.

Further, both straps 30 and 40 are substantially flat and have a predetermined width. The elastic fore strap 40 may be made of an elastic band such as polyurethane, silicone, spandex and latex material. The elastic fore strap 40 may be also made of any other stretchable fabric which stretches in at least one direction.

The back strap 30 may be selected from a non-stretchable fabric such as woven fabric, but not limited thereto. The strap 30 may also be selected from an elastic fabric such as a knitted fabric and a fabric using textured yarns or elastic yarns. The back strap 30 may be preferably made of the same material as the outer sheet 21 of the forehead shield for a simple and attractive appearance.

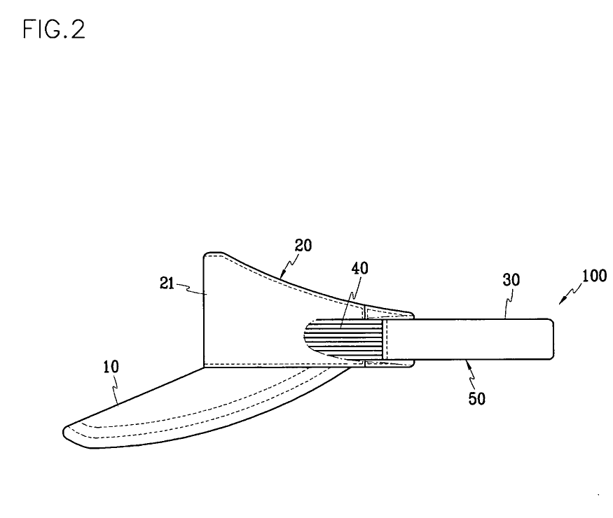

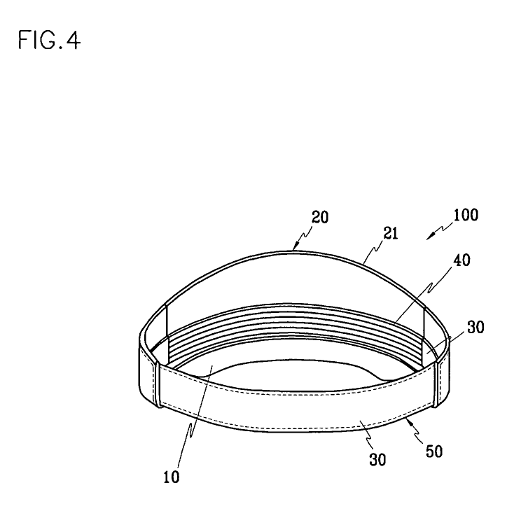

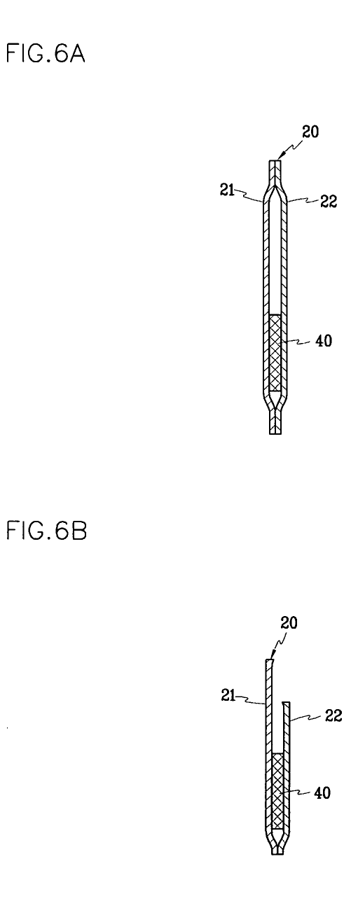

Referring now to FIGS. 2, 4 and 6A-6B, the elastic fore strap 40 will be described in detail. FIG. 2 illustrates the visor cap 100 wherein a portion of the outer sheet 21 of a forehead shield 20 has been broken away to reveal the elastic fore strap 40. FIG. 4 illustrates a view wherein the inner sheet 22 is removed in the visor cap 100. FIGS. 6A-6B are cross-sectional views of examples of a forehead shield 20 in which the elastic fore strap 40 is inserted.

The inner sheet 22 has the same size as that of the outer sheet 21 as shown in FIG. 6A, so the inner sheet 22 is coupled to the outer sheet 21 at both a lower and upper hem. The elastic fore strap 40 may be disposed freely between the outer sheet 21 and the inner sheet 22 of the forehead shield 20. Although it is not shown in the drawings, the strap 40 may also be coupled either of the outer sheet 21 or the inner sheet 22 on one or more spots by an appropriate coupling method such as stitching, adhesion and so forth.

Alternatively, the inner sheet 22 may have a smaller size than that of the outer sheet 21 as shown in FIG. 6B, so the inner sheet 22 is coupled to the outer sheet 21 at a lower hem. In this case, the height of the inner sheet is long enough to cover the elastic fore strap 40. The elastic fore strap 40 is also inserted between sheets 21 and 22.

Further, the elastic fore strap 40 is covered with the forehead shield 20 to be concealed within the forehead shield 20 at rest or when stretched. Therefore, the visor cap 100 has simple appearance. When the elastic fore strap 40 is fixed to the forehead shield 20 on by one or more spots, the elastic fore strap is prevented from shifting from its position to expose, resulting in a simple appearance.

When worn the visor cap 100 allows for different sizes of heads by stretching the elastic fore strap 40 to fit the head of the wearer. The size adjustment is accomplished using elastic fore strap 40. Therefore, the visor cap 100 is adjustable to fit heads of various sizes.

Since the elastic fore strap 40 is pulled toward the forehead of the wearer, it may prevents the visor cap 100 from being stripped off or slipped down. However, the elastic fore strap 40 is not in direct contact with the forehead of the wearer, so the wearer feels wearing comfort due to soft fit of the cap.

In contrast to the conventional visor cap in which the fore part of the sweatband is stitched to a lower hem of the forehead shield around the visor potion, since the sweatband 50 is not fixed on the lower hem of the forehead shield, the visor cap 100 of the present invention may enhance the soft fit of the cap.

In addition, the elastic fore strap 40 is concealed within the forehead shield 20 at rest or when stretched. Accordingly, when the back strap 30 is made of a same material as the outer sheet 21 of the forehead shield 20, the visor cap 100 is constructed of the same material, resulting in a simple attractive appearance.

The visor cap 100 has other advantages in that it may offer enhanced pleasantness due to rapid sweat absorption since it has double absorption construction of the elastic fore strap 40 and the inner sheet 22 of the forehead shield 20.

It should be noted that other kinds of sweatband may be employed although the above description is made referring to FIGS. 1 to 5 in which the sweatband 50 has the fore strap 40 and rear strap 30. For example, a sweatband is formed in shape of a ring of which an entire part is made of an elastic material. The sweatband may be knitted by elastic or non-elastic yarns. The sweatband may also be weaved using elastic yarns such as polyurethane, silicone, spandex and latex material or any other elastic yarns.

It will be apparent to those skilled in the art that various modifications and variations can be made to the device of the present invention without departing from the spirit and scope of the invention. The present invention covers the modifications and variations of this invention provided they come within the scope of the appended claims and their equivalents.

1. A visor cap for being worn by a wearer, comprising:
a visor portion for blocking sunlight;
a forehead shield coupled to the visor portion, having an outer sheet and an inner sheet;
a sweatband formed in shape of a ring to encircle a head of the wearer, wherein a fore part of the sweatband is disposed between the outer sheet and the inner sheet of the forehead shield, the sweatband comprising:
a back strap having a first end and a second end, and
an elastic fore strap, disposed between the outer sheet and the inner sheet of the forehead shield, having a first end and a second end,
wherein the first end and the second end of the back strap are connected to the first end and the second end of the elastic fore strap, respectively, to encircle a head of the wearer.

2. (canceled)

3. The visor cap as recited in claim 21, wherein the elastic fore strap is concealed within the forehead shield.

4. The visor cap as recited in claim 3, wherein the elastic fore strap is concealed within the forehead shield when it is stretched.

5. The visor cap as recited in claim 1, wherein the sweatband is not fixed to the forehead shield.

6. The visor cap as recited in claim 1, wherein the sweatband is fixed to the forehead shield on one or more spots.

7. The visor cap as recited in claim 1, wherein the sweatband is elastic.

8. The visor cap as recited in claim 21, wherein the back strap is elastic.

9. The visor cap as recited in claim 1, wherein the outer sheet or the inner sheet of the forehead shield is comprised of one or more pieces.

10. The visor cap as recited in claim 1, wherein the inner sheet has the same size with that of the outer sheet.

11. The visor cap as recited in claim 1, wherein the inner sheet has a smaller size than that of the outer sheet.

12. A cap, comprising:
a visor portion;
a forehead shield attached to the visor portion, the forehead shield having an outer sheet and an inner sheet; and
a sweatband formed in a ring to encircle a head of a wearer, the sweatband having an elastic forepart and a non-elastic back strap,
wherein the elastic forepart is enclosed between the outer sheet and the inner sheet of the forehead shield.
